(19) United States
(12) Patent Application Publication
(10) Pub. No.: US 2013/0318863 A1
(43) Pub. Date: Dec. 5, 2013

(54) CORE-SHELL MAGNETIC COMPOSITE AND APPLICATION ON PRODUCING BIODIESEL USING THE SAME

(71) Applicants: Jo-Shu CHANG, Taichung City (TW); Dang-Thuan TRAN, Tainan City (VN); Chien-Chang HUANG, Tainan City (TW); Ching-Ling CHEN, Kaohsiung City (TW)

(72) Inventors: Jo-Shu CHANG, Taichung City (TW); Dang-Thuan TRAN, Tainan City (VN); Chien-Chang HUANG, Tainan City (TW); Ching-Ling CHEN, Kaohsiung City (TW)

(21) Appl. No.: 13/488,697

(22) Filed: Jun. 5, 2012

(51) Int. Cl. C10L 1/00; B01J 23/745; B01J 21/08; B01J 31/02; B01J 21/00; C12N 11/14

(52) U.S. Cl. 44/307; 502/100; 435/176; 502/232; 502/158; 502/338

(57) ABSTRACT

A core-shell magnetic composite is disclosed, which includes: a magnetic core; a shell containing a protective layer and a porous layer, wherein the protective layer is coated on a surface of the magnetic core and the porous layer is the outmost layer of the shell; and a hydrophobic functional group grafted to the shell. In addition, the core-shell magnetic composite can be bound to a lipase to act as a transesterification catalyst. The present invention also relates to a method for producing biodiesel.

### BACKGROUND OF THE INVENTION

1. Field of the Invention

The present invention relates to a core-shell magnetic composite and applications using the same, especially to a core-shell magnetic composite applicable for combining with lipase to produce biodiesel, and biodiesel production by using lipase immobilized on a core-shell magnetic composite.

2. Description of Related Art

Currently, enzymes are used on an industrial scale as catalysts in the processing treatment of various crude materials. However, such processing treatment is only cost-effective when the enzymes are recyclable and capable for re-use. For achieving this purpose, the enzymes should be separated from the processing solution, for which general practice provides attaching enzymes to filterable or separable carriers.

Industrial enzymes are usually amphiphilic molecules, i.e. possessing both hydrophilic and hydrophobic properties, which include for example, lipases and phospholipases. When such an enzyme is dispersed in an emulsion, it moves to and accumulates at the interface between the water phase and the oil phase, wherein the hydrophobic region inserts into the oil phase while the hydrophilic region inserts into the water phase.

In the above enzymes, lipases are usually used for its ability to modify triglyceride and lipid. Lipases may catalyze various kinds of triglyceride conversions, such as hydrolysis, esterification, and transesterification, wherein the hydrolysis may be used to convert oil into free acid, and the transesterification may be used to convert vegetable oil or animal oil into biodiesel.

However, the transesterification for biodiesel has many limitations at present. For example, the water and methanol contents should not be too high. When the water content is too high, lipase may start to catalyze oil hydrolysis reaction, and the methanol concentration may decrease due to dilution effect at the same time, thereby decreasing the transesterification efficiency. In addition, when the methanol concentration is too high, lipase may be poisoned and lose its activity. Therefore, the methanol needs to be added fractionally during the process to prevent a high methanol content, thus increasing processing complexity.

Thus, what is needed in the art is to develop a composite which may combine various kinds of lipases, with improved tolerance to water and methanol contents and may be recovered rapidly and reused repeatedly, to advantageously improve the yield and efficiency of related processes.

### SUMMARY OF THE INVENTION

An object of the present invention is to provide a core-shell magnetic composite, which employs a magnetic core coated with a shell, wherein the surface of the shell is mesoporous to have a higher surface area for coupling with enzyme. Furthermore, the surface of the shell is grafted with a hydrophobic group to serve as a bridging group to bind enzyme. The hydrophobic group not only facilitates the binding of a hydrophobic enzyme to the shell but also improves the binding affinity of enzyme, such that the core-shell magnetic composite bound to enzyme may be recovered and reused repeatedly.

To achieve the above object, an aspect of the present invention provides a core-shell magnetic composite, which includes: a magnetic core; a shell containing a protective layer and a porous layer, wherein the protective layer is coated on a surface of the magnetic core and the porous layer is the outmost layer of the shell; and a hydrophobic functional group grafted to the shell.

Commercial magnetic particles which are readily available on the market may be used as the magnetic core of the invention. The surface of the magnetic core is coated with a compact protective layer using the sol-gel process well known in the art, then a porous layer is formed to cover the surface of the protective layer, and finally a hydrophobic functional group is grafted to the porous layer. Accordingly, a hydrophobic region of an enzyme is attracted by the hydrophobicity of the hydrophobic functional group and affixed to the shell surface effectively, while the active region is exposed to achieve catalysis.

In a preferred embodiment of the present invention, the core-shell magnetic composite further comprises a lipase, wherein the hydrophobic functional group is connected to the lipase and the shell. The source of the lipase is not particularly limited, and a readily available commercial lipase or a lipase from a bacterial, a fungi, or an animal, such as Pseudomonas, Candida, Burkholderia, Aspergillus, Rhizopus, porcine pancreas, and so on, may be used. In addition, the enzyme to be bound to the surface of the magnetic core is not limited to a lipase. Similar enzymes or enzymes having a hydrophobic region on its surface are also suitable for use.

The magnetic core of the present invention consists essentially of Fe₃O₄ and thus it may be recovered easily by a magnet for reuse. Besides, the protective layer is not particularly limited, and preferably a compact silicon dioxide (SiO₂) layer, which may protect the magnetic core from damage during the catalysis reaction to reduce likelihood of low recovery rate or avoid producing impurities that reduce reaction efficiency. On the other hand, the porous layer is not particularly limited, and preferably an amorphous silicon dioxide layer which is mesoporous, i.e. the pore size between about 2 nm to about 50 nm, thereby providing a higher surface area. For example, the surface area and the pore volume of the core-shell magnetic composite (not yet combined with an enzyme) are about 150 m²/g to 250 m²/g and about 0.15 m³/g to 0.25 m³/g respectively.

In the core-shell magnetic composite of the present invention, the hydrophobic functional group needs to include a long carbon chain with about 6 to 30 carbon atoms in which a bond between two carbon atoms may be a single bond, a double bond or a triple bond, and the long carbon chain may be interposed by a nitrogen atom to form a primary, secondary or tertiary amine, or ammonium, or interposed by an oxygen atom, a sulfur atom or the like to form the other functional groups. Because the hydrophobic functional group is grafted to the outer surface of the shell, an enzyme having a hydrophobic region may be easily attracted by the hydrophobic long carbon chain of the hydrophobic functional group. Alternatively, a hydrogen bond may be formed between a nitrogen or oxygen atom of the enzyme and the hydrophobic functional group. In addition, an enzyme having a negative charge may also be attracted by the positively-charged ammonium of the hydrophobic functional group.

For example, the hydrophobic functional group may comprise a C₁₀₋₃₀ alkyl ammonium and a siloxanyl connected to the C₁₀₋₃₀ alkyl ammonium, and be bonded to the amorphous silicon dioxide layer through the siloxanyl. Preferably, the hydrophobic functional group may comprise a C₁₄₋₂₄ alkyl ammonium. In this case, the long carbon chain of the hydrophobic functional group may be combined with the hydrophobic region of the enzyme by a van de Waals force. The hydrophobic functional group having a positively-charged ammonium may be combined with the enzyme having a negative charge through an ionic bond formed by the attraction between the positive and negative ions. Accordingly, the means of the combination between the enzyme and the hydrophobic functional group is not merely limited to a covalent bond.

In a preferred exemplary embodiment of the present invention, [3-(trimethoxysiliy)propyl]octadecyl dimethyl ammonium chloride is employed to treat the surface of the shell of the core-shell magnetic composite to remove the methyl group from the trimethoxysiliy so that the oxygen can be bonded to the silicon of silicon dioxide.

Another object of the present invention is to provide a method for producing biodiesel, wherein a core-shell magnetic composite having a lipase immobilized on its surface is used as a catalyst, which may maintain a good catalytic activity in an environment of relatively high water and methanol content. Therefore, the processes for removing a free acid and water as well as the fractional addition of methanol are not required. Meanwhile, the catalyst can be recovered with a magnet for reuse, thus simplifying the process.

To achieve the above object, another aspect of the present invention provides a method for producing biodiesel comprising the following steps: performing a transesterification at an oil/alcohol molar ratio of 1/14.5 to 1/3 and a water content of 10 wt % to 47 wt % using a catalyst, wherein the catalyst is the above-mentioned core-shell magnetic composite combined with the lipase.

The method for producing biodiesel may further comprise the following step: recovering the catalyst with a magnet. The catalyst can be recovered rapidly with a magnet due to the presence of the magnetic core and the superior binding affinity between enzyme and the core-shell magnetic composite.

In the method for producing biodiesel of the present invention, the type of the alcohol is not particularly limited, and preferably a C₁₋₆ alkyl alcohol, such as methanol, ethanol, or the like. When methanol is used as the reactant, the oil/alcohol molar ratio may be 1/14.5 to 1/3 depending on the user's requirements. The amount of the catalyst may range from 5 wt % to 30 wt % based on the oil content, for example, depending on the content of the reactant (i.e. oil and alcohol). The water content may vary depending on the source of oil, and a good yield may be obtained when the water content is from 10% to 47% (based on the total weight of oil and water). The transesterification is preferably performed at a temperature of 25° C. to 60° C. to ensure a conversion rate of above 70%.

In summary, the present invention employs a magnetic core coated with a shell, wherein the surface of the shell is a porous structure bound with a hydrophobic functional group to have a higher binding affinity with an enzyme. In addition, the core-shell magnetic composite of the present invention can be combined with a lipase to form a catalyst. The catalyst may be used for oil hydrolysis to transfer the oil into a free acid as a raw material in chemical industry. Alternatively, the catalyst may also be used directly for transesterification to transfer the oil into a fatty acid methyl ester as biodiesel or other raw materials in chemical industry. Furthermore, such catalyst has a very high tolerance to water and methanol, and may be separated and recovered by a magnetic force after the reaction for reuse.

### BRIEF DESCRIPTION OF THE DRAWINGS

FIG. 1 shows the result of transesterification at different temperatures in Example 1.

FIG. 2 shows the result of reuse test in Example 2.

### DETAILED DESCRIPTION OF THE PREFERRED EMBODIMENT

The making and using of the embodiments of the disclosure are discussed in detail below. It should be appreciated, however, that the embodiments provide many applicable inventive concepts that can be embodied in a wide variety of specific contexts. The specific embodiments discussed are merely illustrative, and do not limit the scope of the disclosure.

### Synthetic Example

### Preparation of the Core-Shell Magnetic Composite

A commercial Fe₃O₄ magnetic particle was used as the magnetic core, and tetraethyl orthosilicate (TEOS) was used as the source of silicon dioxide (SiO₂). In a sol-gel method, TEOS was hydrolyzed to coat the surface of the magnetic core with a compact silicon dioxide (SiO₂) protective layer. In this Example, the source and method for forming silicon dioxide are not limited to the above-mentioned, and it can be readily appreciated by a person of ordinary skill in the art that other sources and methods for forming the compact silicon dioxide (SiO₂) protective layer may also be used.

Then, the coating of a porous silicon dioxide layer was performed in the presence of the surfactant and ammonia water. In this Example, hexadecyl trimethyl ammonium bromide (HTAB) was used as a cationic surfactant, and the porous silicon dioxide layer thus formed was mesoporous, wherein the surface area and the pore volume were measured as being 202 m²/g and 0.2 m³/g respectively. However, it can be readily appreciated by a person of ordinary skill in the art that other methods for forming the porous silicon dioxide layer may also be used.

After that, dimethyloctadecyl [3-(trimethoxysilyl)propyl] ammonium chloride was used as the source of the hydrophobic functional group to be grafted onto the porous silicon dioxide layer. However, other sources and methods for forming the hydrophobic functional group may be used.

### Immobilization of Lipase

The above prepared core-shell magnetic composite was suspended in a solution containing the lipases. After the lipases were entirely caught and immobilized by the magnetic carrier, the core-shell magnetic composite having immobilized lipase was recovered by a powerful magnet, and freeze-dried for storage.

### Comparative Synthetic Example

The same method for preparing the core-shell magnetic composite as described in the Synthetic Example was employed, except that the surface of the core-shell magnetic composite was not grafted with a hydrophobic functional group. Subsequently, immobolization of a lipase was performed as described above.

### Example 1

### Transesterification at Different Temperature

The core-shell magnetic composite having immobilized lipase in the Synthetic Example was used as a catalyst. The effect of reaction temperature on production yield of biodiesel was investigated under the following conditions: catalyst amount: 11 wt %, high class olive oil/methanol molar ratio: 1/4, water content: 10 wt %, stirring speed: 600 rpm, and reaction time: 30 hours.

The obtained biodiesel was analyzed with a gas chromatography (Shimadzu GC-14B; column: Agilent DB-17ht). The temperature ramp-up program was set with an initial column temperature of 150° C. for 2 minutes, and the temperature was increased to 250° C. at a ramp-up rate of 10° C./min and then maintained at 250° C. for 5 minutes.

The biodiesel conversion rate was measured according to the following equation, wherein the biodiesel formed by transesterification with sodium hydroxide was defined as 100%.

Conversion rate (%)=(Signal area of biodiesel formed by catalysis with heterogeneous basic catalyst)/(Signal area of biodiesel formed by transesterification with sodium hydroxide)

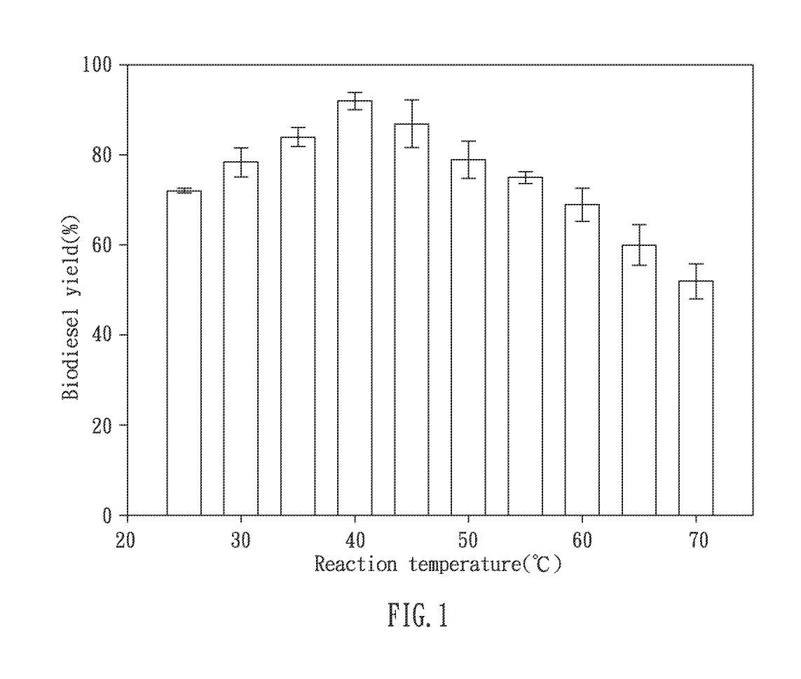

The results in FIG. 1 show that the maximum biodiesel yield (92±1.9%) was obtained at a reaction temperature of 40° C. Also, the lipase of the invention exhibited a conversion rate of above 70% over a wide range of operating temperature from 25 to 60° C.

### Example 2

### Reuse Test

Likewise, the core-shell magnetic composite having immobilized lipase in the Synthetic Example was used as a catalyst.

The tests were performed at reaction temperatures of 25° C. and 40° C. with reaction times of 30 and 40 hours respectively. Other reaction conditions were the same: catalyst amount of 12.5 wt %, the high class olive oil/methanol molar ratio of 1/3, water content: 2.2 wt %, the stirring speed: 500 rpm.

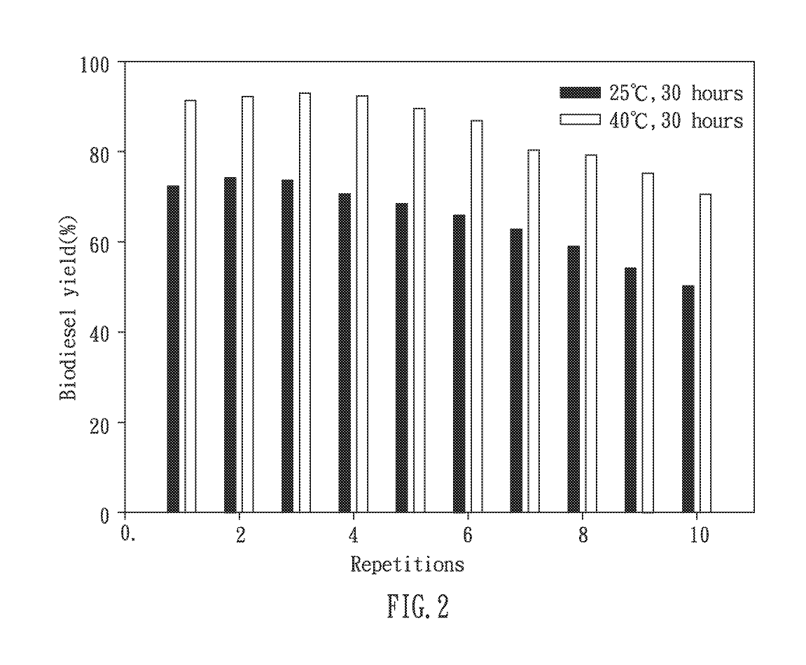

FIG. 2 shows that the biodiesel yield decreased slowly from 72.3% to 50.3% in the condition of 25° C. for 30 hours while the biodiesel yield decreased slowly from 92.3% to 70.8% in the condition of 40° C. for 40 hours, using the core-shell magnetic composite having immobilized lipase in the Synthetic Example as catalyst.

This result indicates that the core-shell magnetic composite having immobilized lipase had not only superior transesterification ability, but also remarkable operating lifespan.

### Example 3

### Versatility Test of Oil Source

Likewise, the core-shell magnetic composite having immobilized lipase in the Synthetic Example was used as a catalyst, and high-class olive oil, commercially available soybean oil and sunflower oil were used as the oil sources. In addition, the water and methanol tolerances were also tested during the experiment.

The reaction conditions were: catalyst amount of 6.67 wt %, oil/methanol molar ratio of 1/9.5 to 1/14.5, water content of 31 wt % to 47 wt %, stirring speed of 600 rpm, reaction temperatures of 40° C. to 50° C., and reaction time of 40 hours. The result was shown in Table 1.

TABLE 1

| | | | | | | fatty acid |
|---|---|---|---|---|---|---|
| | | | | | stirring | methyl esters |
| | methanol/oil | water | catalyst | Temp. | speed | conversion |
| Oil source | molar ratio | (wt %) | (wt %) | (° C.) | (rpm) | rate (%) |
| olive oil | 9.5-14.5 | 30.5-46.8 | 6.67 | 40-50 | 600 | 90-95 |
| soybean oil | 9.5-14.5 | 30.5-46.8 | 6.67 | 40-50 | 600 | 90-95 |
| sunflower oil | 9.5-14.5 | 30.5-46.8 | 6.67 | 40-50 | 600 | 90-95 |

reaction time: 40 hours

Table 1 shows that the core-shell magnetic composite having immobilized lipase had superior transesterification ability for all of the high-class olive oil to commercially available edible oil as the oil sources, indicating its high versatility. In addition, the biodiesel yield shows a high reading of above 90% when the water content is 46.8 wt % and the oil/methanol molar ratio is 1/14.5, indicating its superior tolerance to water and methanol.

### Example 4

### Reusability

The core-shell magnetic composites having immobilized lipase in the Synthetic Example and the Comparative Synthetic Example were used as a catalyst.

The tests were performed at a reaction temperature of 25° C. for a reaction time of 30 hours. The other common conditions were: catalyst amount of 11 wt %, high class olive oil/methanol molar ratio of 1/4, water content of 10 wt %, and the stirring speed of 600 rpm. The fatty acid methyl ester (FAME) conversion rates were shown in Table 2.

TABLE 2
FAME conversion rate (wt %)

| | Times | | | | | | | | | |
|---|---|---|---|---|---|---|---|---|---|---|
| Catalyst | 1 | 2 | 3 | 4 | 5 | 6 | 7 | 8 | 9 | 10 |
| Synthetic | 72.3 ± 3.2 | 75.5 ± 1.9 | 74.3 ± 2.8 | 70.2 ± 4.2 | 71.4 ± 5.4 | 73.5 ± 3.2 | 69.7 ± 2.2 | 67.1 ± 2.9 | 65.6 ± 3.7 | 60.1 ± 4.1 |
| Example | | | | | | | | | | |
| Comparative | 65 ± 1.2 | 41 ± 2.7 | 23 ± 1.5 | 10 ± 0.7 | 0 | ND | ND | ND | ND | ND |
| Synthetic | | | | | | | | | | |
| Example | | | | | | | | | | |

ND: Not detected

As shown in Table 2, the core-shell magnetic composite of the Synthetic Example had better reusability than that of the Comparative Synthetic Example in that hydrophobic functional group was used to combine the lipase and the core-shell magnetic composite.

In summary, in order to separate the lipase from the products of the transesterification reaction for recovering and reuse, the present invention develops a core-shell magnetic composite, wherein a magnetic core is coated with a shell having a two-layer structure. The shell has a compact layer and a porous layer, wherein the compact layer is used to protect the magnetic core while the porous layer is used to increase the surface area. In addition, a hydrophobic functional group is further grafted to the shell, thereby increasing the binding affinity and efficiency with an enzyme. In addition, the core-shell magnetic composite combined with a lipase may serve as a catalyst for transesterification of biodiesel. Even under conditions of a high methanol/oil molar ratio of 9.5 to 14.5, a high water content of 31 wt % to 47 wt %, and a reaction temperature of 40° C. to 50° C., the biodiesel yield can still reach 90% to 95%. This indicates that the core-shell magnetic composite having a lipase has a great catalytic ability as well as superior water and methanol tolerance. Therefore, when low-quality oil (such as waste cooking oil) and crude microalgae lipid having high water content are adopted for the biodiesel raw material, the biodiesel synthesis can be carried out without the need to remove fatty acid and water. In addition, it is not necessary to add methanol fractionally during the transesterification reaction, thereby simplifying the process. Also, the catalyst can be separated and recovered rapidly by a magnetic force after the reaction for reuse.

The making and using of the embodiments of the disclosure are discussed in detail below. It should be appreciated, however, that the embodiments provide many applicable inventive concepts that can be embodied in a wide variety of specific contexts. The specific embodiments discussed are merely illustrative, and do not limit the scope of the disclosure.

What is claimed is:

1. A core-shell magnetic composite, comprising:
a magnetic core;
a shell containing a protective layer and a porous layer, wherein the protective layer is coated on a surface of the magnetic core and the porous layer is the outmost layer of the shell; and
a hydrophobic functional group grafted to the shell.

2. The core-shell magnetic composite of claim 1, wherein the protective layer is a compact silicon dioxide (SiO₂) layer.

3. The core-shell magnetic composite of claim 1, wherein the porous layer is an amorphous silicon dioxide layer.

4. The core-shell magnetic composite of claim 3, wherein the hydrophobic functional group comprises: a C₁₀₋₃₀ alkyl ammonium and a siloxanyl connected to the C₁₀₋₃₀ alkyl ammonium, wherein the siloxanyl is bonded to the amorphous silicon dioxide layer.

5. The core-shell magnetic composite of claim 1, wherein the hydrophobic functional group is formed by treating a surface of the shell with [3-(trimethoxysiliy)propyl]octadecyl dimethyl ammonium chloride.

6. The core-shell magnetic composite of claim 1, wherein the magnetic core consists essentially of Fe₃O₄.

7. The core-shell magnetic composite of claim 1, wherein the porous layer is mesoporous.

8. The core-shell magnetic composite of claim 1, further comprising: a lipase, wherein the hydrophobic functional group is connected to the lipase and the shell.

9. A method for producing biodiesel, comprising:
performing a transesterification at an oil/alcohol molar ratio of 1/14.5 to 1/3 and a water content of 10 wt % to 47 wt % using a catalyst, wherein the catalyst is the core-shell magnetic composite of claim 8.

10. The method for producing biodiesel of claim 9, wherein the alcohol is a C₁₋₆ alkyl alcohol.

11. The method for producing biodiesel of claim 9, wherein the catalyst is added in an amount ranging from 5 wt % to 30 wt %.

12. The method for producing biodiesel of claim 9, wherein the transesterification is performed at a temperature of 25° C. to 60° C.

13. The method for producing biodiesel of claim 9, further comprising: recovering the catalyst with a magnet.
